(19) United States
(12) Patent Application Publication
(10) Pub. No.: US 2025/0186362 A1
(43) Pub. Date: Jun. 12, 2025

(54) WIPE WITH NUMBING ELEMENTS FOR USE IN THE TATTOOING PROCESS

(71) Applicant: Mike DeVries, Simi Valley, CA (US)

(72) Inventor: Mike DeVries, Simi Valley, CA (US)

(21) Appl. No.: 18/534,349

(22) Filed: Dec. 8, 2023

(51) Int. Cl. A61K 9/70; A61K 8/02; A61K 8/41; A61K 8/73; A61K 31/167; A61K 47/38; A61Q 17/00

(52) U.S. Cl. CPC A61K 9/7007; A61K 8/0208; A61K 8/416; A61K 8/731; A61K 31/167; A61K 47/38; A61Q 17/005

(57) ABSTRACT

A wipe for use in the tattooing process has palliative materials impregnated within it which are adapted to provide preventative care to the skin of a patient being tattooed and is manufactured from a material adapted to absorb excess tattoo ink.

### BACKGROUND OF THE INVENTION

### 1. Field of the Invention

The present invention relates generally to skin care wipes and more particularly to a disposable wipe for use in the tattoo industry which includes pain relief, sanitizing and moisturizing formulations, which results in a soothing effect for the user.

### 2. General Background and State of the Art

Tattooing is the process of the introduction of colored inks into the dermis layer of skin to permanently color the skin. The process involves using a tattoo machine for the controlled application of the colored inks to the dermis layer of a patient's skin, by repeatedly perforating the epidermis layer of skin with controlled punctures by needles coated in ink. Once punctured, the skin cells smear the ink from the surface of the needles, which essentially stains these cells with the desired pigments.

A tattoo is a permanent body modification made by inserting pigment or ink into the layers of skin to change the skin's normal pigmentation, typically for aesthetic or decorative purposes. During the tattooing process the ink is placed under a person's skin using a needle. This results in irritation and damage to the skin and can be painful to the person being tattooed. The skin is repeatedly punctured during the tattooing process and, as a result, the person being tattooed suffers a risk of infection. An infection can occur during the tattooing process due to equipment which has not been properly disinfected. Other sources of infection can include impregnating ink too deep in the patient's skin, overworking an area of the skin or skin breaks during the healing process.

It will be understood that present invention is not restricted to being used concurrently while ink is being applied to the skin. The wipe of the present invention can also be used after the tattooing process is complete and, in that instance, will help in the healing and after-care of the patient. The invention can also be used in other settings. For example, the wipe can be used to care for common household injuries, such as when a child might fall down and suffer a skin abrasion. Similarly, the invention can be used in the athletic field, where athletes may become injured and broken skin requires treatment.

The use of needles to puncture the skin can be more than just simple irritation to the person who is having the tattoo applied. In some instance the process can be very painful to the person whose skin in being tattooed. The skin becomes inflamed and tender, causing more pain. A circular feedback loop is set up, which can at times become intolerable. This situation is obviously undesirable. The user may stop the tattoo process entirely. In other instance the person being tattooed can pull away from the tattoo machine in an effort to reduce pain. This can cause the tattoo artist to deviate from the intended pattern leaving an unsightly appearance on the user's skin.

### INVENTION SUMMARY

The wipe of the present invention overcomes the limitations of the prior art by providing a disposable wipe which has various materials impregnated into it to provide preventative and palliative care to the skin of the person being tattooed. In the preferred embodiment, the wipe is impregnated with a pain relief agent and a sanitizer. In another embodiment, the wipe is further impregnated with a moisturizing element for the skin. In other embodiments, the wipe may be impregnated with differing combinations of the foregoing materials. The overall effect of the present invention results in a soothing effect on the skin of the person who is being tattooed.

### BRIEF DESCRIPTION OF THE DRAWINGS

These and other features of this invention will be best understood by reference to the following detailed description of a preferred embodiment of the invention, taken in conjunction with the accompanying drawings, in which:

FIG. 1 illustrates a wipe of the present invention which is contained in a tear-away package.

### DETAILED DESCRIPTION OF THE PREFERRED EMBODIMENTS

In the following description of the preferred embodiment, reference is made to the accompanying drawings that form a part hereof, and in which is shown by way of illustration a specific embodiment in which the invention may be practiced. It is to be understood that other embodiments may be utilized, and changes may be made without departing from the scope of the present invention. In the following description, disclosure of the invention is made with primary reference to a single-use wipe having impregnated materials for skin-care procedures when used by a person receiving a tattoo. It will be apparent to those of skill in the art, however, that the wipe of the present invention can also be used with other items. The particular arrangements described are intend only for explanatory purposes of a specific example or embodiment of the preset invention and are not intended to be limiting in any manner.

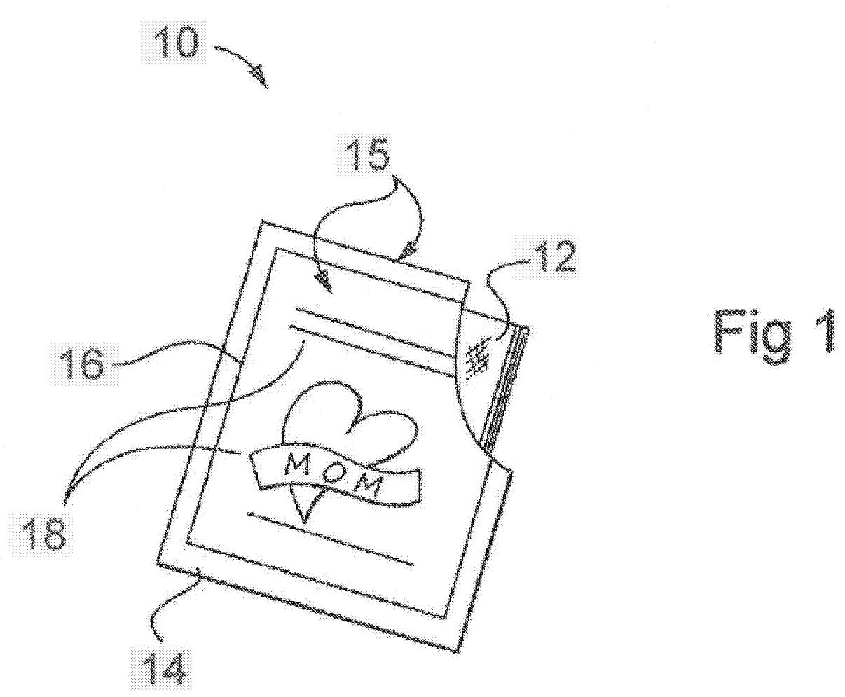

Referring now to FIG. 1, a single use tattoo-enhancing wipe bundle 10 may include a wipe 12 sealed in a tear-open package 14. In FIG. 1, a single corner of the package 14 is shown removed to reveal the wipe 12. The tear-open package 14 has a front and rear plastic sheet 15 sealed around the periphery 16 to contain a square or oblong folded wipe 12 therein. The folded wipe 12 may be a woven or nonwoven absorptive fabric. The plastic sheets 15 may be printed on the outside with identifying information 18, and the front and rear plastic sheets 15 may include a light block such as foil or paper to shield the material of the towelette from both light and humidity.

It is to be understood that the tear-open package shown in FIG. 1 is not intended to limit the scop of the present invention but is for illustrative purposes only. It will be understood by those of skill in the art that the wipe of the present invention can be packaged in a manner which is different from what is shown in FIG. 1. For example, more than one wipe can be included in a bundle, or the wipes may be placed in a dispenser such as that shown in Patent Application Publication No. 2022/0183515 A1, instead of a tear-away package.

A wipe, as used herein, refers to a general fabric. The general fabric maybe disposable but does not need to be as it could be washed and re-used. embodiments of the towelette may be offered in a wide variety of shapes and sizes while adhering to the spirit of the disclosure. The wipe contemplated herein may be reusable after a wash, but preferably is a single use wipe. If an embodiment of the wipe is single use, the wipe may be dispensed in a single foil pack, a soft pack, or a canister, among other methods. The purpose of the dispensing method of being sealed is to prevent the towelette and solution located therein from exposure to the air and atmosphere. The construction and preferred material for wipe are discussed below.

The wipe 12 is impregnated with materials which are intended provide palliative and preventive care to the skin of the person being tattooed. Throughout this disclosure, the person whose skin is being tattooed is referred to as a “patient.” This is not, however, meant to indicate the present invention can only be used in a medical setting. The present invention can be used with all types of tattoos, including tattoos which are solely for cosmetic or decorative procedures. The present invention can also be used to treat other breaks in the skin, such as common household injuries or abrasions. Moreover, the use is not limited to persons being tattooed. For example, athletes who become incurred and suffer skin breaks can use the wipe of the present invention to help treat injuries which include skin breaks or punctures.

The materials which are impregnated in the preferred embodiment of the present invention include materials which have a pain relief and sanitizing function. The impregnated materials may be emulsified, for example, by polyglyceryl-4 laurate to provide a suitable consistency for improved dispersion over the skin. While the applicant does not wish to be bound to a particular theory, it is believed that the oil and water improve the color transparency of the skin to enhance the color and definition of the underlying tattoo pigment. A humectant such as glyceryl and a superabsorbent polymer such as sodium, polyacrylate may he added to the wipe 12 to enhance the efficacy of device.

A patient may experience pain during the tattooing process. Pain can be caused because the tattooing process involves a small needle repeatedly piercing the skin in order to inject ink in a desired location below the epidermis. The needle and ink can often irritate a person's skin, causing irritation, inflammation, and pain. These results are obviously undesirable.

One method of reducing pain is impregnating a product which numbs the skin and relieves pain into the wipe. In the preferred embodiment, the pain relief material used is lidocaine. In one embodiment, the lidocaine is diluted to a strength of 4%. Other dilutions can also be used. For example, it has been found that a dilution as low as 1% or as high as 12% can also be effective. Other materials such as Benzocaine, Novocain, Pramoxine or other similar material can also be used with the wipe of the present invention. In other instances, more than one type of pain reliever is impregnated into the wipe.

A second element which is impregnated in the preferred embodiment of the wipe of the present invention is a sanitizer. The sanitizer is used to keep the skin of the patient in a generally clean or sterile state and prevent infections or other contamination of the small wounds caused by the tattoo machine. In the preferred embodiment, the sanitizer material which is used is benzalkonium chloride, which is commonly known as BZK.

It should be noted that the use of a disposable wipe is advantageous as opposed to simply using creams of liquids on the skin of the patient. A tattoo artist will typically need to remove excess ink which is deposited by a tattoo machine during the tattooing process. Consequently, the tattoo artist can use the wipe to remove excess ink while at the same time providing the skin-care benefits of the present invention. It is therefore desirable that the material which is used for the wipe has the ability to soak up the ink used in the tattooing process.

An alternative embodiment of the present invention also includes a moisturizing element which is impregnated in the wipe. The moisturizing element further treats the skin and provides relief. One such moisturizing element can be aloe vera. Other types of moisturizers may include hyaluronic acid, dimethicone, glycerin, propylene glycol, lanolin, or mineral oil. The use of the various elements, including pain reliever, sanitizer, and moisturizer results in a soothing effect on the skin of a patient who is using the wipe of the present invention.

The construction and constituency of the material which is used for the formation of the wipe will now be discussed. In the preferred embodiment, the wipe is manufactured from viscose. Viscose is a semi-synthetic type of rayon fabric made from wood pulp that is often used as a silk substitute, as it has a similar drape and smooth feel to the luxury material. The term “viscose” refers specifically to the solution of wood pulp that is turned into the fabric. Viscose is the preferred material because of its soft, pliable nature, and the presence of the wood fibers in it. It is also less expensive than other materials. The wood fibers in viscose provide a scaffold which can be used to retain the impregnated materials which are discussed above. Viscose is also very absorbent and can absorb up to 400 percent of its own weight in moisture. The viscose of the present invention has been modified to provide further absorptive capabilities This is useful for removing excess ink from the user's skin, as noted above. The process lifts leftover ink so the artist can see what is being done to create better art by effectively lifting the ink off properly. All while numbing the area with pain relief for longer sessions and better retention.

Although the wipe of the preferred embodiment is manufactured from viscose, it will be apparent to those of skill in the art that other materials can also be used, as is discussed above.

The composition disclosed herein contains a variety of elements, one of which is water. There are different types of water, such as distilled, de-ionized, filtered water, and the like, but any type of water will do and is not distinguished with regard to the present disclosure.

Thus, it is apparent that there has been provided, in accordance with the invention, a disposable wipe for use in the tattooing process. While this specification describes particular embodiments of a wipe which has sanitizing and pain relief capabilities, it will be apparent that those of ordinary skill can devise variations of the present invention without departing from the overall inventive concept. While the invention has been described in conjunction with specific embodiments thereof, it is evident that many alternatives, modifications, and variations will be apparent to those skilled in the art in light of the foregoing description. While features or functions have been described with respect to one embodiment and perhaps not others, it is understood and intended that features from one embodiment will be combined with features from other embodiments to yield further embodiments. Accordingly, it is intended to embrace all such alternatives, and the scope of the present invention is limited only by the following claims.

What is claimed is:

1. A wipe for use in the tattooing process, said wipe having impregnated therein palliative materials adapted to provide preventative care to the skin of a patient being tattooed and being manufactured from a material adapted to absorb excess tattoo ink.

2. The wipe of claim 1, wherein said palliative materials include a pain reliever and a sanitizer.

3. The wipe of claim 2, wherein said pain reliever comprises lidocaine.

4. The wipe of claim 3, wherein said lidocaine has a strength in the range of 1% to 10% by weight.

5. The wipe of claim 4 wherein said lidocaine has a strength of substantially 4% by weight.

6. The wipe of claim 2, wherein said sanitizer comprises benzalkonium chloride.

7. The wipe of claim 1, wherein said palliative materials further include a moisturizing element.

8. The wipe of claim 1 wherein said wipe is manufactured from a flexible fabric.

9. The wipe of claim 8 wherein said flexible fabric comprises viscose.

10. The wipe of claim 1, wherein said wipe is disposable.

11. A disposable wipe for use in the tattooing process, said wipe being manufactured from a flexible absorptive material and having impregnated therein lidocaine and benzalkonium chloride.

12. The disposable wipe of claim 1I wherein said absorptive material comprises viscose.

13. The disposable wipe of claim 12 wherein said lidocaine is present in a strength in the range of 1% to 10% by weight.

14. The wipe of claim 13 wherein said lidocaine has a strength of substantially 4% by weight.

15. The disposable wipe of claim 11, further comprising a moisturizing element impregnated therein.

16. A disposable wipe for use in the tattooing process, said wipe being manufactured from viscose and having impregnated therein lidocaine, benzalkonium chloride and a moisturizing element, and wherein said lidocaine is present in a strength in the range of 1% to 10% by weight.

17. The wipe of claim 4 wherein said lidocaine has a strength of substantially 4% by weight.
